(19) United States
(12) Patent Application Publication
(10) Pub. No.: US 2003/0120147 A1
(43) Pub. Date: Jun. 26, 2003

(54) Method for determining the total body fat content of a subject by analysis of MR images

(75) Inventors: Rainer Kuth, Herzogenaurach (DE); Thomas Rupprecht, Uttenreuth (DE); Maren Wagner, Erlangen (DE)

Correspondence Address: SCHIFF HARDIN & WAITE, 6600 SEARS TOWER, 233 S WACKER DR, CHICAGO, IL, 60606-6473, US

(73) Assignee: Siemens Aktiengesellschaft

(21) Appl. No.: 10/327,801

(22) Filed: Dec. 19, 2002

(30) Foreign Application Priority Data
Dec. 20, 2001 (DE) 10163015.8

(51) Int. Cl. A61B 5/05

(52) U.S. Cl. 600/410

(57) ABSTRACT

In a method for the high-precision determination of the body fat content, whereby the test subject is measured with an MR scanner tuned to the fat resonant frequency and the resulting MR images are analyzed as to the areas therein representing fat.

### BACKGROUND OF THE INVENTION

1. Field of the Invention

The present invention is directed to a method for determining the total body fat content of a subject.

2. Description of the Prior Art

Determination of the body fat content in the human body currently is made nearly exclusively on the basis of the body mass. The precision that is achieved may be acceptable for an absolute measurement in many cases, however, this method is too imprecise for a relative measurement in an individual patient when a therapy is to be monitored. The reason for this is that the body mass is subject to relatively large short-term fluctuations due to the ingestion and elimination of food, water absorption in the tissue and the significantly fluctuating protein content in the body. There are therapies involving the elimination of up to 3 liters of water in only a few hours as a side effect, and conversely a person may drink up to 6 liters of water per day.

The determination of the body mass is extraordinarily important for the diagnosis in a number of diseases. Too high a fat content, for example, represents a high risk for diabetes as well as for vascular and joint conditions.

### SUMMARY OF THE INVENTION

An object of the present invention is to provide a method for determining the total body fat content that exhibits a high absolute precision and a very exact, relative precision per patient.

This object is achieved in accordance with the invention in such a method wherein the test subject is measured with an MR scanner tuned to the fat resonant frequency. A volume measurement of the fat over the entire body as well as—preferably—an integral measurement over one or more slices can ensue.

As a result of the inventive measurement of the fat content using an MR scanner, wherein, in contrast to the “normal” use of such an MR scanner, it is not tuned to the water frequency but is “detuned” to the somewhat different fat frequency, the subcutaneous fat content that is especially critical in conjunction with risk diseases can be very exactly determined.

The slices can be selected on the basis of anatomical landmarks in the integral measurement for determining the relative fat content. Anatomical landmarks are, for example, the spinal column for a sagittal measurement and the navel for an axial measurement.

The fat determination can be especially quickly and simply implemented when the measurement ensues with a TrueFisp sequence or other sequences as well. The measurement time for acquiring a tomogram with such a TrueFisp sequence amounts to only approximately 1 second.

When, for the slice-by-slice acquisition of the entire body, the test subject is moved through the MR scanner by displacing the support table and the fat layer is segmented in the individual images and the fat content is electronically calculated from the total volume, then this requires a measurement time of only 3 minutes based on slice thicknesses of approximately 2 cm in a sequence of, for example, 90 axial sections from head to foot.

### DESCRIPTION OF THE DRAWINGS

FIG. 1 is an axial tomogram given a patient having normal weight.

FIG. 2 is an axial tomogram given an overweight patient.

FIG. 3 is the tomogram of FIG. 2, wherein the fat has been segmented.

### DESCRIPTION OF THE PREFERRED EMBODIMENTS

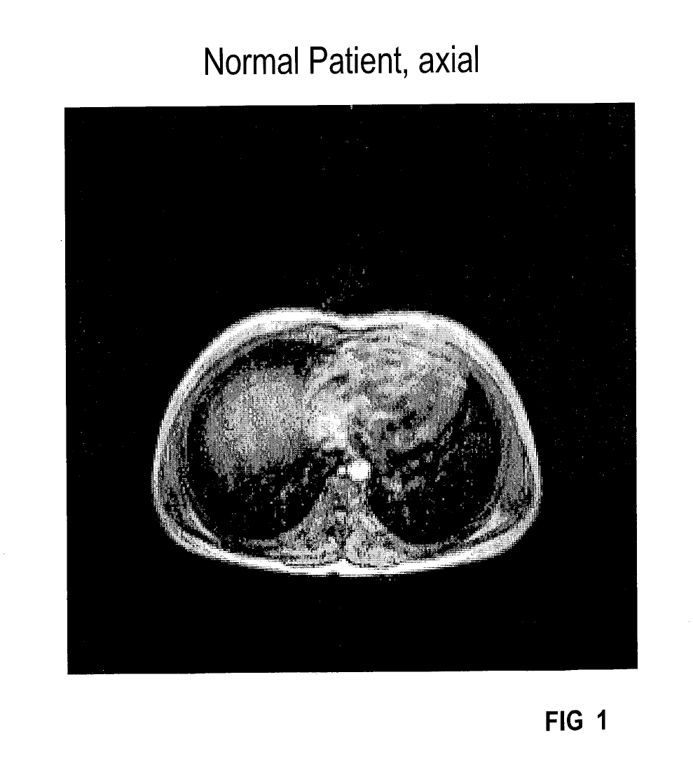

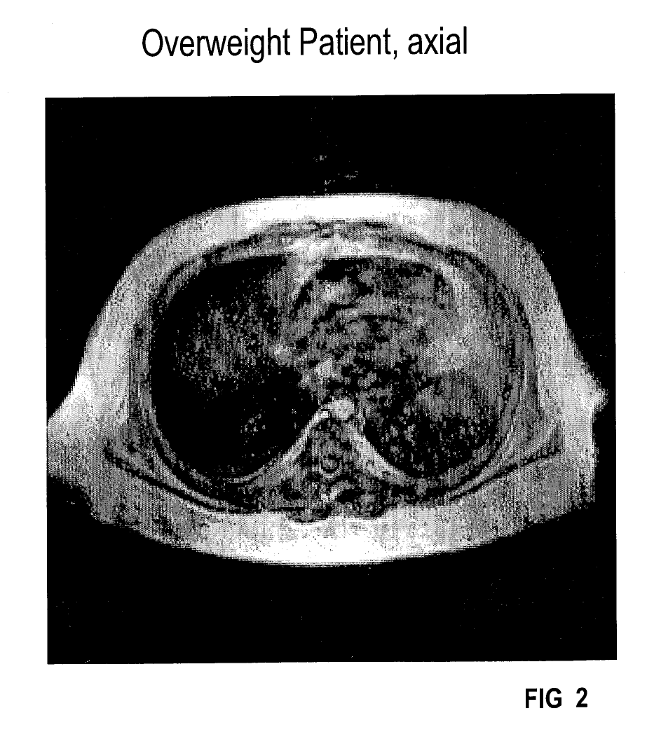

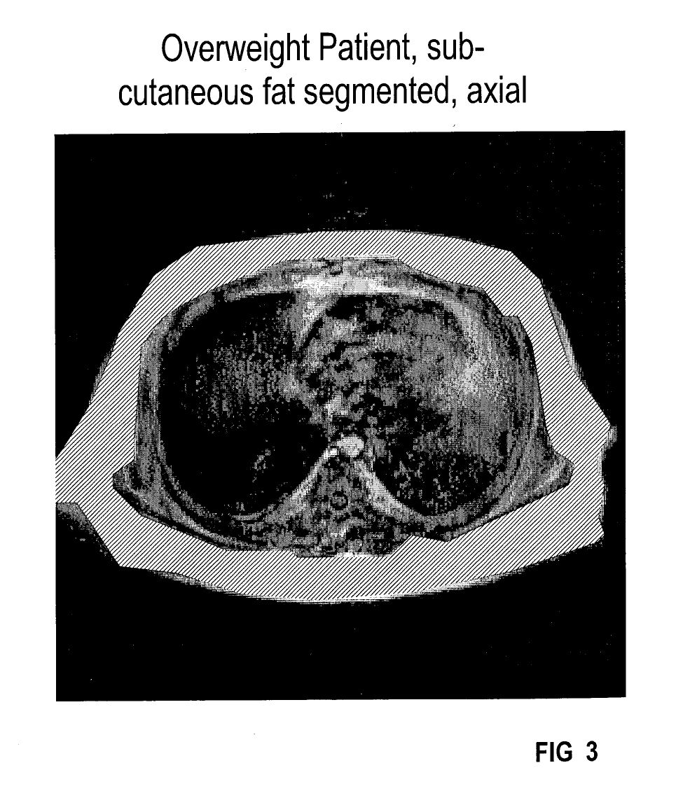

By comparing the two images in FIGS. 1 and 2, one can very clearly see how highly elevated the subcutaneous fat layer is in overweight patients compared to those having normal weight. The fat content in the individual slices can be very exactly determined from the area of this tomogram acquired using an MR scanner tuned to the fat resonant frequency. The fat content determination can ensue very simply by electronically segmenting and encircling the layer of the high fat content according to FIG. 3 followed by a calculation algorithm. The overall fat mass in the body can be determined significantly more exactly from the addition of the fat volumes than is the case in known methods.

Although modifications and changes may be suggested by those skilled in the art, it is the intention of the inventors to embody within the patent warranted hereon all changes and modifications as reasonably and properly come within the scope of their contribution to the art.

We claim as our invention:

1. A method for high-precision determination of the total body fat content of a subject, comprising the steps of:
obtaining magnetic resonance data from a subject with a magnetic resonance scanner tuned to the resonant frequency of fat; and
electronically analyzing said magnetic resonance data to determine the total fat content of said subject therefrom.

2. A method as claimed in claim 1 wherein the step of obtaining said magnetic resonance data comprises obtaining a volume measurement of magnetic resonance data over an entirety of the body said subject.

3. A method as claimed in claim 1 wherein the step of obtaining said magnetic resonance data comprises obtaining magnetic resonance data from said subject in at least one slice, and making an integral measurement over said at least one slice to determine said total body fat content.

4. A method as claimed in claim 3 comprising selecting said slice dependent on anatomical landmarks in said magnetic resonance data.

5. A method as claimed in claim 3 comprising obtaining said magnetic resonance data using a TrueFisp sequence.

6. A method as claimed in claim 3 comprising obtaining said magnetic resonance data from a plurality of slices of said subject by moving said subject through said magnetic resonance scanner for a slice-by-slice acquisition of said magnetic resonance data, and electronically segmenting portions in the respective slices representing fat and electronically calculating said total body fat content from a totality of said regions in all of said slices.
